(19) United States
(12) Patent Application Publication
(10) Pub. No.: US 2019/0142461 A1
(43) Pub. Date: May 16, 2019

(54) TROCAR SEAL PROTECTOR ASSEMBLY WITH PLEATS

(71) Applicant: 5RMED TECHNOLOGY(CHENGDU) CO., LTD., Chengdu (CN)

(72) Inventor: Moshu ZHU, CHENGDU (CN)

(73) Assignee: 5RMED TECHNOLOGY(CHENGDU) CO., LTD.

(21) Appl. No.: 16/249,953

(22) Filed: Jan. 17, 2019

Related U.S. Application Data
(63) Continuation of application No. PCT/CN2017/093600, filed on Jul. 20, 2017.

(30) Foreign Application Priority Data
Aug. 2, 2016 (CN) 201610630357.7

(51) Int. Cl. A61B 17/34

(52) U.S. Cl. CPC A61B 17/3439; A61B 2017/347; A61B 17/3498

(57) ABSTRACT

The invention discloses a trocar seal protector assembly comprising improved pleats. Said protector assembly comprises a proximal opening, a distal end and central axis. Said distal end comprises a plurality of pleats, each of which including a pleat-ridge, a pleat-valley and a pleat-wall extending from the pleat-ridge to the pleat-valley. The plurality of pleats are arranged in a dish shape around the central axis and define a distal aperture, the boundary of the distal aperture being formed by a wavy annular circuit, which is completely on a cylindrical surface or completely on a frustum surface. Taking the central axis as the center, make arbitrary cylindrical surface and all pleats in the adjacent area of the distal aperture to be intersected. intersection line of which is a complete wavy annular line. The proximal opening of the protector assembly further includes a boss and a cylindrical wall extending from the proximal end to the distal end. The seal membrane and the protector assembly do not interfere with each other after the inversion, and the bending accumulation and entanglement of the protector assembly and the seal membrane can be reduced, thereby reducing the frictional resistance after the inversion.

### CROSS-REFERENCE TO RELATED APPLICATIONS

This application is a continuation of International Patent Application No. PCT/CN20171093600 with a filing date of Jul. 20, 2017, designating the United States, now pending, and further claims priority to Chinese Patent Application No. 201610630357.7 with a filing date of Aug. 02, 2016. The content of the aforementioned applications, including any intervening amendments thereto, are incorporated herein by reference.

### TECHNICAL FIELD

The present invention relates to a minimally invasive surgical instrument, and in particular, to an improved trocar seal protector assembly.

### BACKGROUND OF THE PRESENT INVENTION

A trocar is a surgical instrument, that is used to establish an artificial access in minimally invasive surgery (especially in rigid endoscopy). Trocars comprise in general a cannula and an obturator. The surgical use of trocars generally known as: first make the initial skin incision at the trocar insertion site, then insert the obturator into the cannula, and then together they facilitated penetration of the abdominal wall through incision into the body cavity. Once penetrated into the body cavity, the obturator is removed, and the cannula will be left as access for the instrument get in/out of the body cavity.

In rigid endoscopy surgery, it is usually necessary to establish and maintain a stable pneumoperitoneum for the sufficient surgical operation space. The cannula comprises a sleeve, an outer body, a seal membrane (also known as instrument, seal) and a duck bill (also known as closure valve). Said cannula providing a channel for the instrumentation in/out of the body cavity said outer body connecting the sleeve, the duck bill and the seal membrane into a sealing system; said duck bill normally not providing sealing for the inserted instrument, but automatically closing and forming a seal when the instrument is removed; said seal membrane accomplishing a gas-tight seal against the instrument when it is inserted.

In a typical endoscopic procedure, it is usually set up 4 trocars (access) i.e. 2 sets of small diameter cannula (normally 5 mm in diameter), and 2 sets of, large diameter cannula (normally 10˜12 mm in diameter). Instruments, in general passing through a small cannula, are only for ancillary works; herein one large cannula as an endoscope channel, and the other large cannula as the main channel for surgeon to perform surgical procedures. Through said main channel thereof, 5 mm diameter instruments used in approximately 80% of the procedure, and said large cannula used in approximately 20% of the procedure; furthermore, 5 mm instruments and large diameter instruments need to be switched frequently. The small instruments are mostly used, so that the sealing reliability of which is more important. The large instruments, are more preferably used in a critical stage of surgery (Such as vascular closure and tissue suturing), therein switching convenience and operational comfort are more important.

Taking a 12 mm diameter trocar in the clinical application (usually 12.8˜12.9 mm in diameter) as an example: when a 5 mm diameter instrument is used, it is approximately considered that the hoop force, generated by the deformation of the sealing lip (i.e. the local material that forms the center hole of the seal membrane), ensures a reliable seal for the inserted instruments. When a large diameter instrument (12 mm diameter anastomat or 10 mm diameter titanium applier) is inserted, the sealing lip and its adjacent area are expanded to the appropriate size and wrapped around the outer surface of the instrument, which results in greater frictional resistance between the seal membrane and the instrument. Said large frictional resistance is normally easy to cause the seal membrane damage, the seal inversion, poor comfort of performance, even result, in cannula insecurely fixed on the patient's abdominal wall etc., so that the performance of trocar is seriously affected. The simplest way to reduce the frictional resistance is reducing the thickness of said seal membrane, however, it is inevitable that the seal membrane is easily torn or punctured by the inserted instrument.

U.S. Pat. No. 5,342,315 discloses a seal protector assembly including four pie-shaped leaf portions, said protector assembly comprising at least two protector members positioned in axial alignment with one another in a facing relationship. Said protector assembly is for preventing perforations or tears to the seal membrane caused by the inserted instrument, and also can reducing the frictional resistance between the instrument and the seal membrane.

An integrated seal protector assembly with pleats is disclosed in U.S. Pat. No. 7,988,671, 8,257,317, 8,597,251, which device comprises integrated seamless frustoconical shield; said shield comprises a plurality of pleats with peaks and valleys; said shield prevents from damaging the seal membrane by the inserted instruments and prevents the seal membrane from being inverted.

FIG. 1-6 illustrate seal protector assembly 1260 and the seal membrane 1250 disclosed in U.S. Pat. No. 7,988,671. Said seal protector assembly 1260 is generally tubular, comprising a proximal end 1262 and a distal end 1264. Said seal protector assembly 1260 is dimensioned and shaped to be received within, carried on or mounted on, the seal membrane 1250 without interfering with the operation thereof. The proximal end 1262 comprises a flange 1266 that engages a corresponding recess 1256 in the seal 1250. The seal protector assembly 1260 moves or floats in concert with the seal membrane 1250. The distal end 1264 is generally frustoconical-shape and mates with the distal end 1258 of the seal membrane 1250. The frustoconical distal end 1264 comprises a plurality of pleats 1268, which are discussed in terminating in an opening 1270. A cylindrical wall 1272 extends between the proximal end 1262 and the distal end 1264.

Although embodiments are disclosed in U.S. Pat. No. 7,988,671, these embodiments have two features in common: one is that the pleats are generally seamless, and the other is that the shape of pleats are generally frustum. Defining the axis of the seal protector assembly 1260 as the longitudinal axis 1261. FIG. 3 is projection view of the seal protector assembly 1260 from the distal end 1264 to the proximal end 1262 along the axis 1261. The opening 1270 is defined by a complete annular-wavy line 1265. A cross section which is generally perpendicular to said axis 1261 intersects said distal end 1264. The formed intersection line of which is annular-wavy line; or at least in the adjacent area of opening 1270 making a cross section which is generally perpendicular to said axis 1261 to intersect all pleats 1268, the formed intersection line of which must be a complete annular-wavy line, wherein it is called the planar-wavy circuit. The distal end 1264 can be considered as a numerous complete planar annular-wavy circuits stacked along the axis 1261. Generally, the circumference of any one of the planar annular-wavy circuits must be larger than the outer circumference when the largest diameter instrument inserted, wherein all plane wavy circuits are relaxed in various degrees. that is, all of the pleats 1268 are generally relaxed.

Said seal protector assembly 1260 has two main defects. Referring to FIG. 5, the opening 1270 defined by the annular-wavy line 1265 includes a plurality of clearances that make the exposed region 1259 of the distal end 1258 larger. It will be understood by those skilled in the art that when the external instrument is inserted, the exposed area 1259 is easily damaged; especially, when the titanium clamp is inserted, the cutting edge of the titanium clamp is opened to form a U-shaped fork structure, and the U-shaped fork is often automatically guided by the pleats 1268 to the exposed area 1259, thereby increasing the risk of piercing the seal membrane. FIG. 6 shows a schematic diagram of trocar shield 1260 and the seal membrane 1250 when the instrument (coagulation hook) is removed. It will be understood by those skilled in the art that the protector assembly 1260 and the seal membrane 1250 are prone to inversion when certain instruments are applied, or when the instrument is removed from an angle. While the inversion risk of the protector assembly 1260 is also disclosed in U.S. Pat. No. 7,988,671.A protector assembly 1260 in which the proximal end 1262 is relatively rigid and the distal end 1264 is relatively soft is disclosed in U.S. Pat. No. 7,988,671. For example, the seal protector assembly 1260 is composed of a multilayer membrane, said proximal end 1262 comprising more layers while the distal end 1264 less layers, thereby allowing the distal end 1264 to be inverted. Studies have shown that since the distal end 1264 comprises a plurality of pleats 1268, while which possess a strong property of enhancing the bending stiffness of the distal end 1264; when the distal end 1264 is stretched outwardly or rotated inwardly, it is more likely to rotate around the intersection region 1263 where the distal end 1264 intersects the cylindrical wall 1272, instead of bending at a part in the middle of the distal end 1264. It will be understood by those skilled in the art that disclosed in U.S. Pat. No. 7,988,671, when the distal end 1264 is inverted, when the pleat-wall 1267 of the distal end pleat 1268 is rotated inwardly around the intersecting region 1263, the distal end 1264 itself is interfered with each other. No disclosure in U.S. Pat. No. 7,988,671 regarding the self-interference of the distal end 1264 when it is inverted. In addition, in some cases the distal end 1264 itself is bent and then inverted, which in turn tends to cause the sealing membrane distal end 1258 and the curved distal end 1264 to accumulate or intertwine each other, resulting in blockage or abnormally increased frictional resistance.

The invention provides an improved pleated protector assembly that effectively resolves one or more of the aforementioned defects.

### SUMMARY OF PRESENT INVENTION

One object of the invention is to provide a seal protector assembly that minimizes the seal membrane exposed outside the coverage of the protector assembly, and at the same time prevents the seal membrane and the seal protector assembly from jamming after inversion.

In one aspect of the present invention, a seal protector assembly for minimally invasive surgery comprises a proximal opening, a distal end, and a central axis. Said distal end comprises a plurality of pleats, each of which including a pleat-ridge, a pleat-valley and a pleat-wall extending from the pleat-ridge to the pleat-valley. The plurality of pleats are arranged in a dish shape around the central axis and define a distal aperture, the boundary of the distal aperture being formed by an annular-wavy circuit, which is completely on a cylindrical surface or completely on a frustum surface. Take the central axis as the center, make arbitrary cylindrical surface to intersect with all the pleats in the adjacent area of the distal aperture, the intersection line of which is a complete annular-wavy circuit. The proximal opening of the protector assembly further includes a boss and a cylindrical wall extending from the proximal end to the distal end. The pleats extend laterally outward from the distal aperture and intersect the cylindrical wall to form a triangular transition region.

In another aspect of the present invention, a seal protector assembly for minimally invasive surgery comprises a proximal opening, a distal end, and a central axis. Said distal end comprises a plurality of pleats, each of which including a pleat-ridge, a pleat-valley and a pleat-wall extending from the pleat-ridge to the pleat-valley. The plurality of pleats are arranged, in a dish shape around the central axis and define a distal aperture, the boundary of the distal aperture being formed by an annular-wavy circuit, which is completely on a cylindrical surface. Taking the central axis as the center, make arbitrary cylindrical surface to intersect with all the pleats in the adjacent area of the distal aperture, intersection line of which is a complete annular-wavy circuit. The proximal opening of the protector assembly further includes a boss, a cylindrical wall extending from the proximal end to the distal end, and a rib extending from the cylindrical wall and intersecting said pleat-valley; while the pleat-ridge and its adjacent pleat wall are cantilevered shape and disconnect with the cylindrical wall.

In one aspect of the present invention, a seal protector assembly for minimally invasive surgery comprises a proximal opening, a distal end, and a central axis. Said distal end comprises, a plurality of pleats, each of which including a pleat-ridge, a pleat-valley and a pleat-wall extending from the pleat-ridge to the pleat-valley. The plurality of pleats are arranged in a dish shape around the central axis and define a distal aperture, the boundary of the distal aperture being formed by an annular-wavy circuit, which is completely on a cylindrical surface. Taking the central axis as the center, make arbitrary cylindrical surface to intersect with all the pleats in the adjacent area of the distal aperture, intersection line of which is a complete annular-wavy circuit. The proximal opening of the seal protector assembly further includes a mounted flange that extends laterally outward from the distal aperture and intersects the mounted flange. The depth of the pleats is gradually reduced, when which extend laterally outward.

In one aspect of the present invention, a seal protector assembly for minimally invasive surgery comprises a proximal opening, a distal end, and a central axis. Said distal end comprises a plurality of pleats, each of which including a pleat-ridge, a pleat-valley and a pleat-wall extending from the pleat-ridge to the pleat-valley. The plurality of pleats are arranged in a dish shape around the central axis and define a distal aperture, the boundary of the distal aperture being formed by an annular-wavy circuit, which is completely on a cylindrical surface. Taking the central axis as the center, make arbitrary cylindrical surface to intersect with all the pleats in the adjacent area of the distal aperture, intersection line of which is a complete annular-wavy circuit. All or the portion of the pleat-ridges or the pleat-valleys includes cuts or slots, in the adjacent area of the distal aperture.

In one aspect of the present invention, a seal protector assembly for minimally invasive surgery comprises a proximal opening, a distal end, and a central axis. Said distal end comprises a plurality of pleats, each of which including a pleat-ridge, a pleat-valley and a pleat-wall extending from the pleat-ridge to the pleat-valley. The plurality of pleats are arranged in dish shape around the central axis and define a distal aperture, the boundary of the distal aperture being formed by an annular-wavy circuit, which is completely on a cylindrical surface. Taking the central axis as the center, make arbitrary cylindrical surface to intersect with all the pleats in the adjacent area of the distal aperture, intersection line of which is a complete annular-wavy circuit. Said proximal opening includes a plurality of cantilevers and slots.

Another object of the invention is to provide a seal membrane assembly, which comprises a seal membrane and a protector assembly, which is dimensioned and shaped to be received within, carried on or mounted on, the seal membrane without interfering with the operation thereof. Said protector assembly may be embedded in the seal membrane or mechanically and adhesively secured to the seal for prevent the center of the seal membrane from puncturing or tearing by the inserted instrument.

Another object of the invention is to provide a seal assembly, which including the above seal membrane assembly, an upper body and an upper cover. Said seal membrane assembly secured between said upper body and said upper cover.

### DESCRIPTION OF THE DRAWINGS

A more complete appreciation of this invention, and many of the attendant advantages thereof will be readily apparent as the same becomes better understood by reference to the following detailed description, where:

FIG. 1: shows a 3D perspective view of the protector assembly in the prior art;

FIG. 2: shows a longitudinal cross-sectional view of the protector assembly in FIG. 1 of the prior art;

FIG. 3: shows a bottom projection view of the protector assembly in FIG. 1 of the prior art;

FIG. 4: shows a longitudinal cross-sectional view of the seal membrane assembly of the seal shield in FIG. 1 of the prior art;

FIG. 5: shows a 3D perspective partial sectional view of the seal, membrane assembly in FIG. 4;

FIG. 6: shows a simulated distorted view of the cannula with the instrument removed in FIG. 5 of the prior art;

FIG. 7: shows a 3D perspective partial sectional view of the cannula in the invention;

FIG. 8: shows top projection view of the seal membrane assembly of cannula in FIG. 2;

FIG. 9: shows a longitudinal cross-sectional view of the seal membrane assembly in FIG. 8;

FIG. 10: shows a 3D perspective partial sectional view of the protector assembly in FIG. 8;

FIG. 11: shows a 3D perspective reserve view of the protector assembly in FIG. 10;

FIG. 12: shows a sectional view along-line 12-12 in FIG. 10;

FIG. 13: shows a sectional view along-line 13-13 in FIG. 10;

FIG. 14: shows a 3D perspective view of the protector assembly in the second embodiment;

FIG. 15: shows a 3D perspective reserve view of the seal shield in FIG. 14;

FIG. 16 shows a sectional view along-line 16-16 in FIG. 14;

FIG. 17 is a sectional view along-line 17-17 in FIG. 14:

FIG. 18: shows a 3D perspective view of the protector assembly in the third embodiment;

FIG. 19: shows a 3D perspective reserve view of the protector assembly in FIG. 18;

FIG. 20 is a sectional view along-line 20-20 in FIG. 19;

FIG. 21 is a sectional view along-line 21-21 in FIG. 19;

FIG. 21 is a sectional view along-line 21-21 in FIG. 19 in the third embodiment;

FIG. 23: shows assembled view of the seal membrane assembly in FIG. 22;

FIG. 24: shows a simulated distorted view of the seal membrane assembly with the large diameter instrument inserted in FIG. 23;

FIG. 25: shows a longitudinal cross-sectional view of the seal membrane assembly in FIG. 24;

FIG. 26: shows a 3D perspective view of the protector assembly in the fourth embodiment;

FIG. 27: shows a magnified view of partial area of the center hole of the protector assembly in FIG. 26;

FIG. 28 shows a sectional view along-line 28-28 in FIG. 19;

FIG. 29: shows a 3D perspective view of the protector assembly in the fifth embodiment;

FIG. 30: shows top projection view of the protector assembly in FIG. 29;

FIG. 31 shows a sectional view along-line 31-31 in FIG. 30;

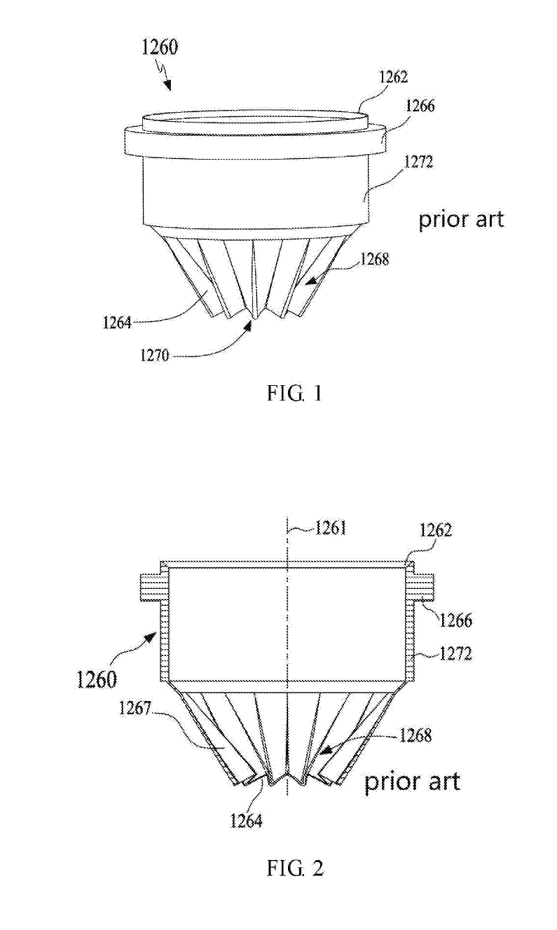

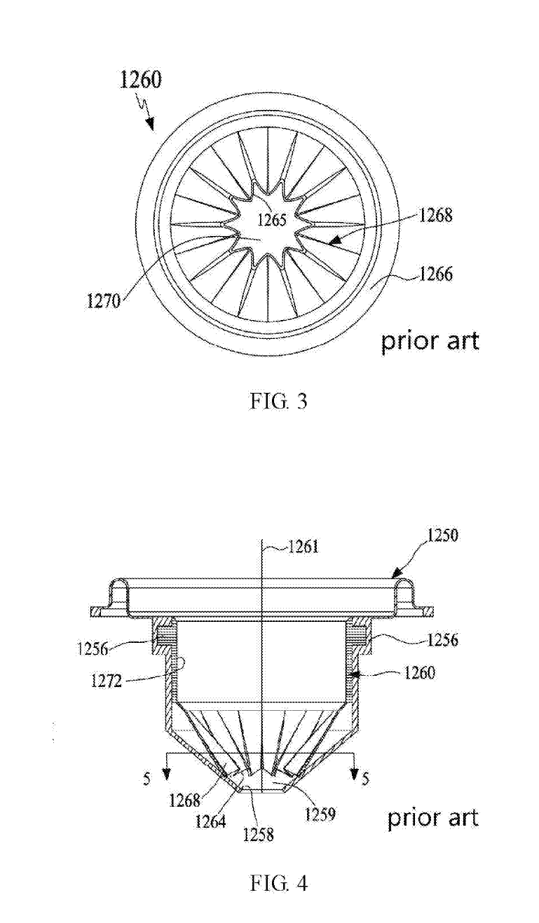

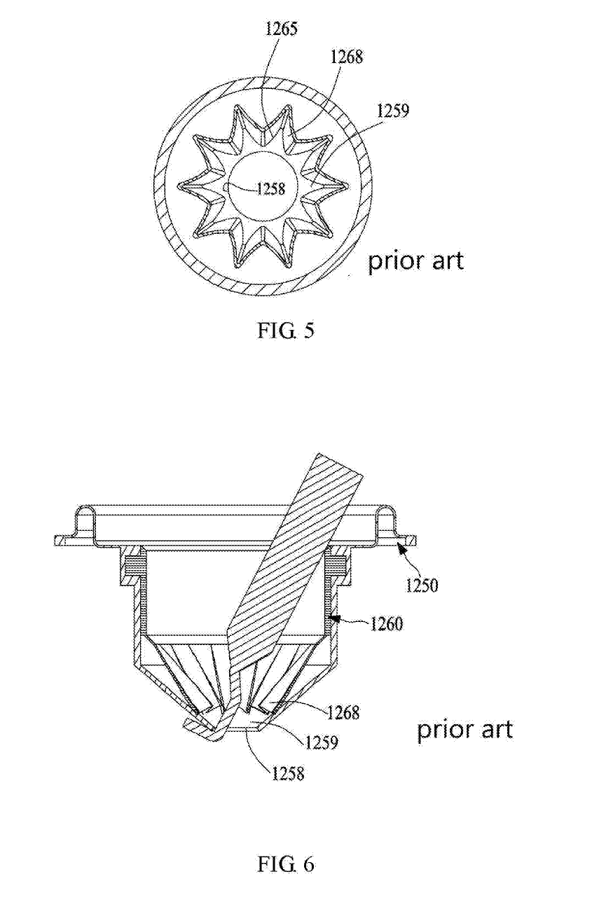

### DETAILED DESCRIPTION OF PREFERRED EMBODIMENTS

Embodiments of the invention are disclosed herein, however, it should be understood that the disclosed embodiments are merely examples of the invention, which may be implemented in different ways. Therefore, the invention is not intended to be limited to the detail shown, rather, it is only considered as the basis of the claims and the basis for teaching those skilled in the art how to use the invention.

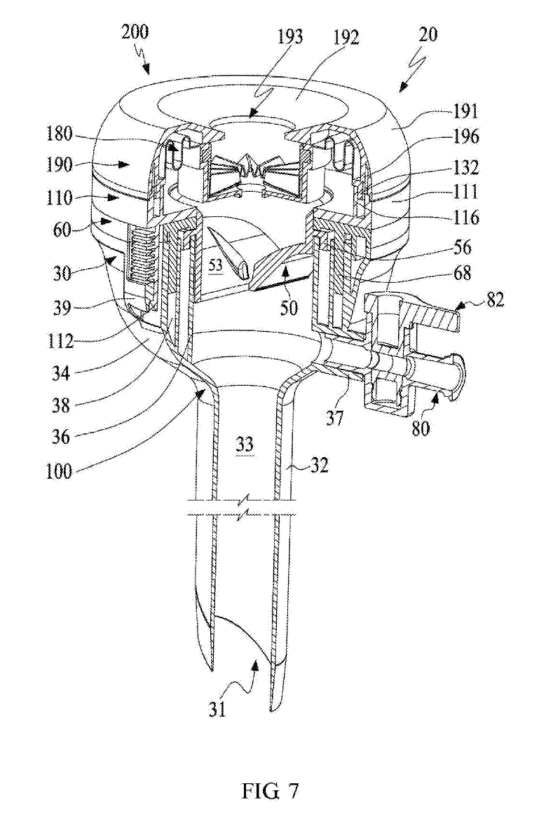

FIG. 7 shows, an overall view of the structure of trocar. A typical trocar comprises an obturator 10 (not shown) and a cannula 20. The cannula 20 comprises an open proximal end 192 and an open distal end 31. In a typical embodiment, said obturator 10 passes through said cannula 20, together they facilitated penetration of the abdominal wall through incision into the body cavity. Once penetrated into the body cavity, the obturator 10 is removed, and the cannula 20 will be left as access for the instrument get in/out of the body cavity. Said proximal end 192 in the external position of the patient and said distal end 31 in the internal position. A preferred cannula 20 can be divided into the first seal assembly 100 and the second seal assembly 200. Locking receptacle 39 in said seal assembly 100 can be locked with snap-in projection 112 in said seal assembly 200. The cooperation of snap-in projection 112 and the locking receptacle 39 can be quick release by one hand. The main purpose is for convenience of taking out tissues or foreign matters from the patient in the surgery. There are multiple ways to implement the quick release connection of said seal assembly 100 and assembly 200. In addition to the structure shown in this embodiment, a threaded connection, a rotary snap-in or other quick lock structure also may be applied. Alternatively, said assembly 100 and assembly 200 can be designed as a structure that can not be split quickly.

FIG. 7 shows the composition and assembly relationship of the first seal assembly 100. The lower body 30 includes an elongated tube 32, which defines the sleeve 33 passed through the distal end 31 and is connected to the outer housing 34. Said lower body 30 comprises an inner wall 36 supporting duck bill seal and a valve bore 37 that communicates with the, inner wall 36. The plunger 82 mounted in the valve body 80, the said two are mounted into said valve bore 37. The flange 56 of the duck bill seal 50 is sandwiched between the inner wall 36 and the lower cover 60. There are various ways of fixing between the lower cover 60 and the lower body 30, such as the interference fit, ultrasonic welding, glue bonding, and snap fastening 4 cylinders 68 of said lower cover 60, in this embodiment, 4 holes 38 of said lower body 30 are adopted to interference fit, so that the duckbill seal 50 is in the compressed state. Said tube 32, said the inner wall 36, said duck bill seal 50, said valve body 80 and said plunger 82 together are comprised the first chamber. Said duck bill seal 50, in this embodiment, is a single-slit, while other types of closure valves may also be used, including flapper valves, multi-silted duck bill valves. When the instrument is passed through said duck bill seal 50, the duckbill 53 will be opened, but it generally does not provide a complete seal against the instrument. When the instrument is removed, said duckbill 53 closed and substantially prevents insufflation fluid from escaping through the first chamber.

FIG. 7 shows the composition and assembly relationship of the second seal assembly 200. The seal membrane assembly 180 is sandwiched between the upper cover 110 and the upper body 190. The proximal end 132 of the seal membrane assembly 180 is secured between the inner ring 116 of the upper cover 110 and the inner ring 196 of the upper body 190. There are various secured ways between the upper cover 190 and the upper body 110, such as the interference fit, ultrasonic welding, glue bonding, and snap fastening. The connection method, shown in this embodiment, is the outer shell 191 of the upper body 190 and the outer shell 111 of the upper cover 110 are secured by ultrasonic welding, so that the proximal end 132 of the seal membrane -assembly 180 is in the compressed state. The center hole 113 of said upper cover 110, said inner ring 116, and said seal membrane assembly 180 together are comprised the second chamber.

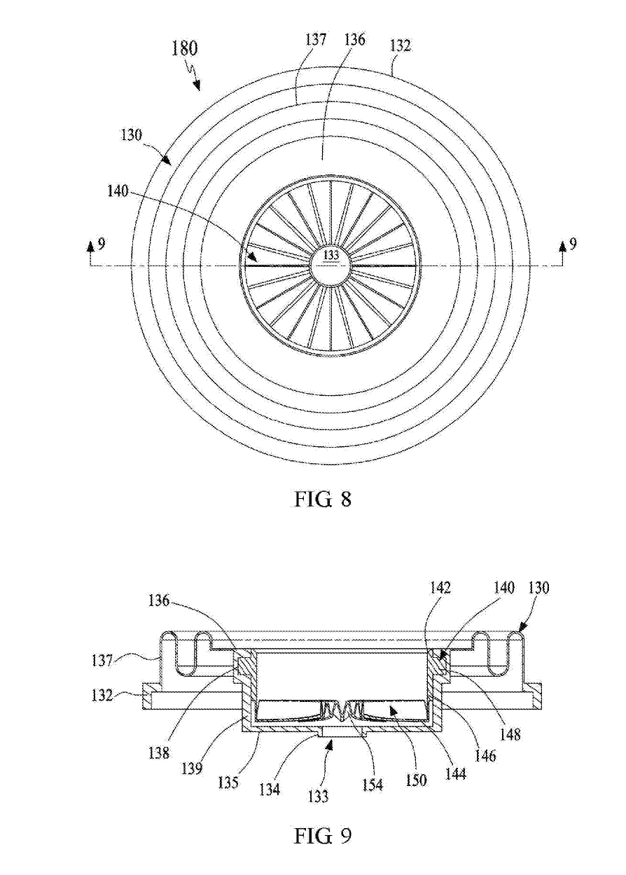

FIG. 8-9 illustrate the composition and assembly relationship of said seal membrane assembly 180, which comprises a seal membrane 130 and a protector assembly 140, which is dimensioned and shaped to be received within, carried on or mounted on, the seal membrane without interfering with the operation thereof The seal protector assembly 140 moves or floats in concert with the seal membrane 130 for prevent the center sealing wall of the seal membrane 130 from puncturing or tearing by the sharp edge of the inserted instrument.

Said seal membrane 130 includes a proximal opening 132, a distal aperture 133, and the sealing wall extending from the distal end to the proximal end, said sealing wall including a proximal surface and a distal surface. Said aperture 133 formed by a sealing lip 134 for accommodating an inserted instrument and forming a gas-tight seal. Said the seal membrane 130 also including the flange 136; The sealing wall 135 has one end connected to the sealing lip 134 and the other end connected to the flange 136; the floating portion 137 has one end connected to the flange 136 and the other end connected to said proximal end 132. Said floating portion 137 including one or several plurality of radial (transverse) pleats, so that the entire seal membrane assembly 180 can float in the assembly 200. Said flange 136 comprises a cylindrical wall 139 and an inner groove 138, said flange is for mounting the protector assembly 140.

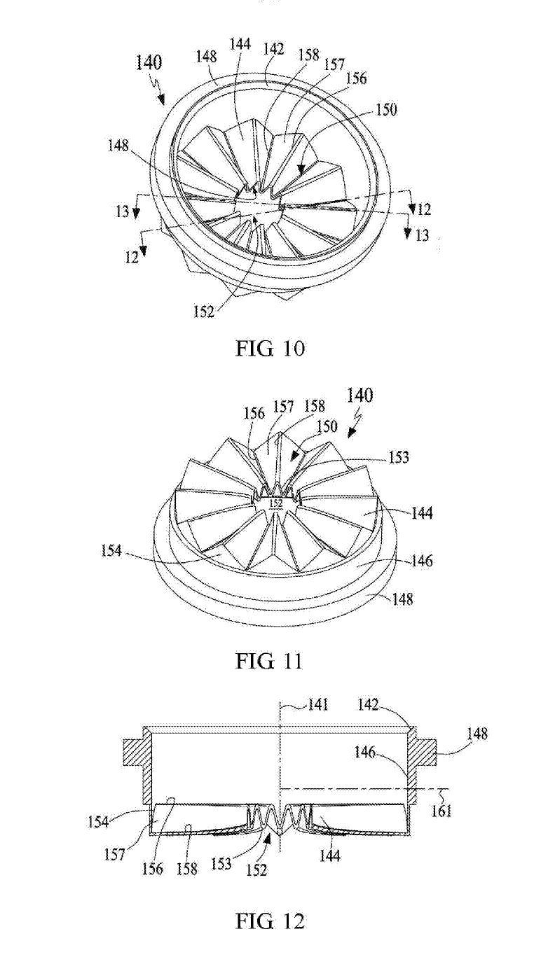

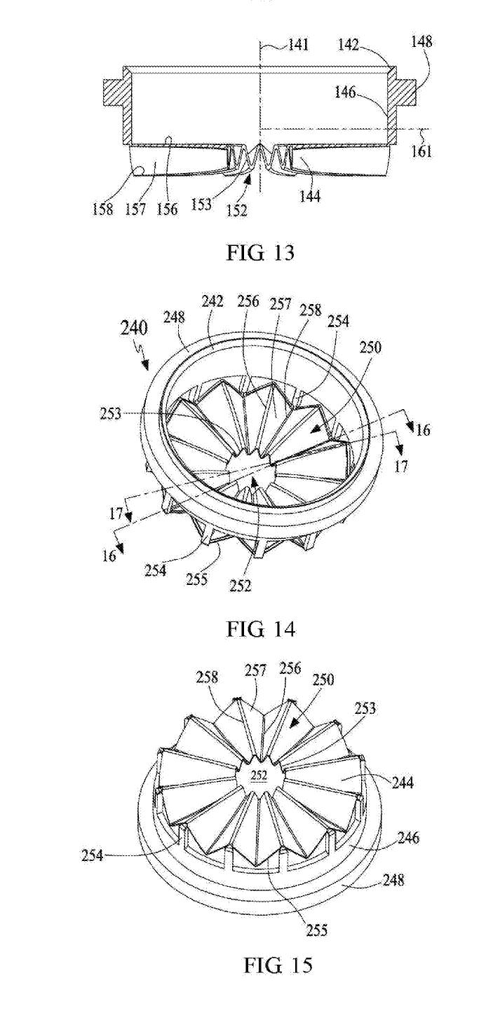

FIG. 10-13 depict the structure and composition of the protector assembly 140 in more detail. Said protector assembly 140 has a central axis 141, which assembly 140 comprises a proximal end 142, a distal end 144 and a cylindrical wall 146 extending from the distal end to the proximal end, which comprises a boss 148, as described above, said cylindrical wall 146 and said boss 148 are shaped and dimensioned to match the cylindrical wall 139 and an inner groove 138 of the seal membrane, so that the protector assembly is embedded in the seal membrane 130.

The distal end 144 is generally dish-shaped and dimensioned to match the aforementioned sealing wall 135. The distal end 144 comprises a plurality of pleats 150, which are arranged in a dish shape around the central axis 141 and, define a distal aperture. More specifically, the distal aperture 152 is defined by a complete annular-wavy line 153, which is formed in such a manner that its annular-wave is substantially on a cylindrical surface. For the convenience of quantification, it is defined when Di is designed as the maximum diameter of the surgical instrument passing through the seal membrane. Taking the axial 141 as the center, draw a cylinder of Di intersects the distal end 144, the area from its intersection line to the distal aperture 152 called the adjacent area of the distal aperture. Taking the axial 141 as the center, draw arbitrary cylindrical surface intersecting said pleats, the intersection line of which is a complete annular-wavy line; or at least taking the axial 141 as the center, draw arbitrary cylindrical surface intersecting the pleats in the adjacent area of the distal aperture, the intersection line of which is a complete annular-wavy circuit, wherein it is referred as a cylindrical-wavy circuit to distinguish it from the planar-wavy circuit described in the background art. The distal end 144 can be regarded as stacked by numerous complete cylindrical-wavy circuit with increasing in, diameter. Generally, the circumference L1 of any cylindrical-wavy circuit is larger than the outer circumference designed for the largest diameter instrument passing through. For example, if the radius designed for the largest instrument passing through is R1, then L1>2*π*R1 (where π=3.14). In this embodiment, there are 12 pleats, while more or less which also can be adopted.

Each of the pleats 150 includes a pleated wall 157 extending from the pleat-ridge 156 to the pleat-valley 158. The pleats 150 extend laterally outward from the annular-wavy line 153 and intersect the cylindrical wall 146 to form a triangular transition region. And said pleats 150 extend laterally outward, and the depth of said pleats remains substantially constant; the measurement method of said pleats depth is: the distance from the pleat-ridge to the pleat-valley along longitudinal axis 141. Defining a transverse plane 161 substantially perpendicular to said axis 141, defining the angle between the pleat-ridge 156 and said transverse plane surface 161 as α, and defining the angle between the pleat-valley 158 and said transverse plane surface 161 as β.

With reference to FIG. 8, the protector assembly 140 in the present embodiment, along longitudinal axis 141 projecting from proximal end to distal end, the distal aperture 152 defined by the pleats 150 is a circle. Compared with the protector assembly disclosed in the background art, the protector assembly 140 can greatly reduce the exposed area of the seal membrane, even if the central area of the seal membrane is barely exposed outside the coverage of the protector assembly. With reference to FIG. 9-13, the pleats 150 are generally dish-shaped, that is, the angle between the pleat-ridge (the pleat-valley) relative to the transverse plane 161 is small or zero; When the instrument is inserted, the cylindrical-wavy circuit is generally stretched, and the pleat-wall 150 is rotated outwardly around the transition zone 154 to a suitable size to accommodate the inserted instrument. When the instrument is removed, in some cases the seal membrane and the protector assembly are inverted; when the diameter of the cylindrical wall 146 is sufficiently large and inversion space are reserved enough, only in the moment of the inversion, the seal membrane and the protector assembly are bent and stacked, resulting in a surge in resistance. As the instrument continues to be pulled out, the pleat-wall 150 is rotated inwardly around the transition zone 154 to an appropriate size. Compared with the protector assembly disclosed in the background art, using the protector assembly 140, the seal membrane and the protector assembly do not interfere with each other after the inversion, and the bending and entanglement of the protector assembly and the seal membrane can be reduced, thereby reducing the frictional resistance after the inversion.

The pleats 150 are generally arranged in a dish-shape and α=β=0°, however, it cannot be, regarded that the angle or β must be zero. The inner diameter of the cylindrical wall 139 is normally 16-20 mm, while the inner diameter of the hole defined by the annular-wavy line 153 is normally 4-6 mm, and when α≥30° or β≥30°, the protector assembly is prone to self-interference when it is inverted. Those skilled in the art can understand that when the pleat-ridge and the pleat-valley have a small angle relative to its pivot point, the protector assembly has a tendency of the pleat convergence and reduce in the moment of the inversion, and the pleated walls 150 around the transition region 154, when the instrument is continuously pulled out, are inwardly rotated to an appropriate size. Normally, 0°≤α≤15°, 0°≤β≤15°.

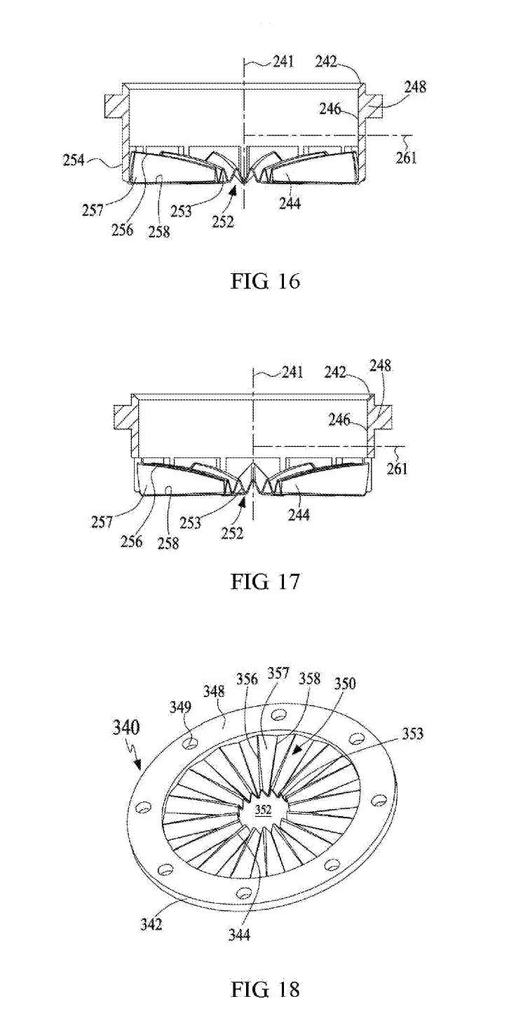

FIG. 14-17 illustrate the structure and composition of the protector assembly 240 in the second embodiment of the invention. Said protector assembly 240 has a central axis 241, which assembly 240 comprises a proximal end 242, a distal end 244 and a cylindrical wall 246 extending from the distal end to the proximal end, which comprises a boss 248, as described above, said cylindrical wall 246 and said boss 248 are shaped and dimensioned to match the cylindrical wall 139 and the inner groove 138 of the seal membrane, so that the protector assembly is embedded in the seal membrane 130.

The distal end 244 is generally dish-shaped and dimensioned to match the aforementioned sealing wall 135. The distal end 244 comprises a plurality of pleats 250, which are arranged in a dish shape around the axis 241 and define a distal aperture. More specifically, the distal aperture 252 is defined by a complete annular wavy circuit 253, which is formed in such a manner that its annular wavy circuit is substantially on a cylindrical surface. Taking the axial 241 as the center, draw arbitrary cylindrical surface intersecting said pleats, the intersection line of which is a complete annular wavy circuit; or at least taking the axial 241 as the center, draw arbitrary cylindrical surface intersecting the pleats in the adjacent area of the distal aperture 252, the intersection line of which is a complete annular wavy circuit, wherein it is referred as a cylindrical-wavy circuit to distinguish it from the planar-wavy circuit described in the background art. The distal end 244 can be regarded as stacked by numerous complete cylindrical wavy rings with increasing in diameter.

Each of the pleats 250 includes a pleat-wall 257 extending from the pleat-ridge 256 to the pleat-valley 258. The pleats 250 extend laterally outward from the annular-wavy line 253 and the pleats 250 portion and the cylindrical wall 246 extend to be intersected. A plurality of cutting slots 255 are included between the pleats 250 and the cylindrical wall 246, the plurality of cutting slots 255 slitting the pleats 250 and the cylindrical wall 246, thereby forming a plurality of ribs 254 to connect the pleats 250 and the cylindrical wall 246. In the embodiment, the pleat peak 256 and pleat wall 257 are severed by 12 cutting slots 255 so as not to intersect the, cylindrical wall 246, and the wave valley 258 is connected to the cylindrical wall 246 by 12 ribs 254. That is, the pleat-ridge 256 of the pleats 250 and most of the pleated walls 257 are in a suspended state. And said pleats 250 extend laterally outward, and the depth of said pleats gradually increases; the measurement method of said pleats depth is: the distance from the pleat-ridge to the pleat-valley along longitudinal axis 241.

The main difference between the second embodiment and the first embodiment is that when the pleats 250 of the second embodiment extend laterally outward, only the pleat-valleys 258 and the cylindrical walls 246 extend to be intersected; while described in the first embodiment, when the pleats 150 extend laterally outward, wherein the pleat-ridge and the pleat-valley simultaneously intersect the cylindrical wall 146. As described above, when the pleats are stretched outwardly or inwardly, the pleats rotate around the transition region of the pleats and the cylindrical wall. However, in the first embodiment, the pleat-ridges and the pleat-valleys rotate in a different arm of force, thereby adding an additional deformation force. It will be understood by those skilled in the art that the pleats 250 of the second embodiment rotate around the intersection of the pleat-valleys 258 and the ribs 254, and the rotary arms are substantially equal, thereby minimizing the additional deformation force.

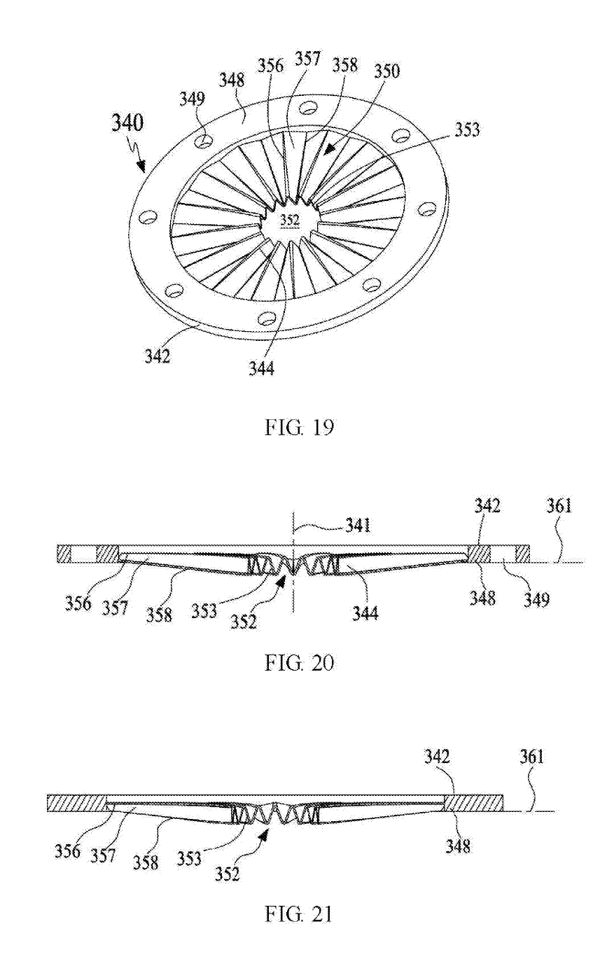

FIG. 18-21 illustrate the structure and composition of the protector assembly 340 in the second embodiment of the invention. Said protector assembly 340 has a central axis 341, which assembly 340 comprises a proximal end 342, a distal end 344. The proximal end 342 includes a flange 348 and an aperture 349 generally uniformed over the flange 348.

The distal end 344 includes a plurality of pleats 350 that are generally arranged in a dish shape around the axis 341 and define a distal aperture 352. In this embodiment there are 16 pleats 350. More specifically, the distal aperture 352 is defined by a complete annular wavy circuit 353. And the annular wavy circuit 353 is formed in such a manner that its annular wave is substantially on a cylindrical surface. It should be understood by those skilled in the art that since the protector assembly has a small depth of pleats and usually has a depth of pleats less than 2 mm, the annular wavy circuit 353 can be formed in such a manner that its hoop wave is completely on a conical surface. Such a slanted annular-wave does not increase the exposed area of the seal membrane covered by the protector assembly to a large extent because the depth of pleats is small. Therefore, it cannot be understood that the annular wavy circuit 353 must be completely on a cylindrical surface; however, an arbitrary cylindrical surface is intersected with the pleats, with the axis 341 as the center, the intersection line of which is a complete annular wavy circuit; or at least an arbitrary cylindrical surface is intersected with the pleats in the adjacent area of the distal aperture 353, with the axis 341 as the center, the intersection line of which is a complete annular wavy circuit. It is referred to herein as a cylindrical-wavy circuit to distinguish it from the planar-wave circuit described in the background art. The distal end 344 can be regarded as stacked by numerous complete cylindrical-wavy circuits with increasing in diameter.

Each of the pleats 350 includes a pleated wall 157 extending between the pleat-ridge 356 and the pleat-valley 358. The pleats 350 extend laterally outward from the annular-wavy line 353 and intersect the flange 348. And said pleats 150 extend laterally outward, and the depth of said pleats gradually decreases; the measurement method of said pleats depth is: the distance from the pleat-ridge to the pleat-valley along longitudinal axis 141. The rate at which the depth of the pleats is decreased can be determined by theoretical calculations and simple tests, wherefore when the large diameter instrument is inserted, the rotary arm of the pleats is consistent when the pleats are fully relaxed.

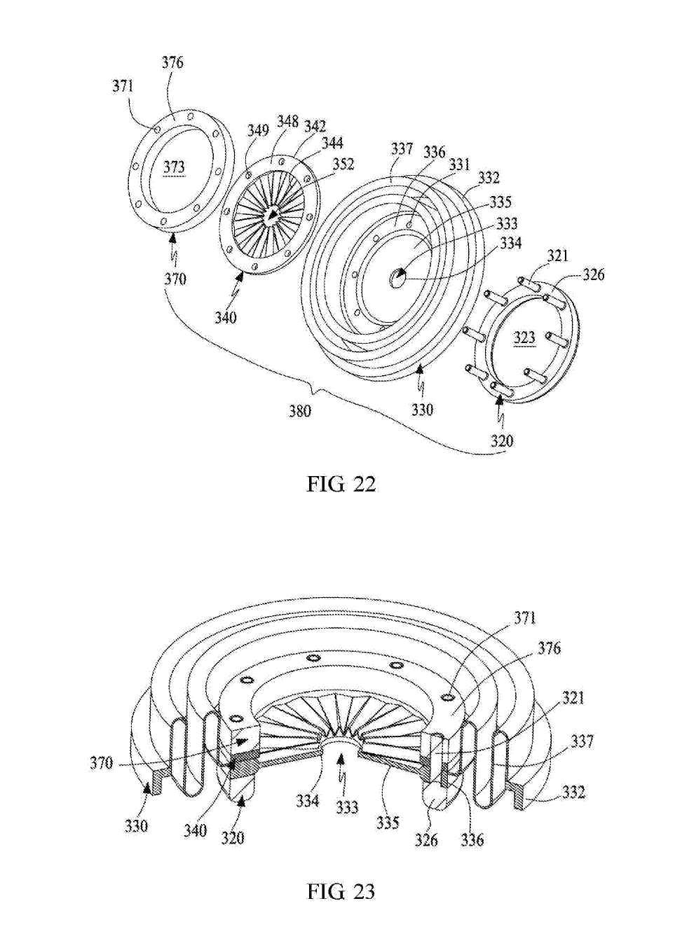

FIG. 22-23 illustrate the structural composition and assembly relationship of the seal membrane 380 comprising the protector assembly 340, said seal membrane comprising a low retainer ring 320 an upper retainer ring 370 and a seal membrane 330, which comprising a proximal opening 332 and a distal aperture 333; which aperture 333 is defined by the sealing, lip 334; the central sealing wall 335 extends from sealing lip 334 to flange 336; the outer floating portion 337 extends, from the flange 336 to the proximal end 332. Said the seal membrane 330 and said protector assembly 340 are sandwiched between the lower retainer ring 320 and the upper retainer ring 370. Moreover, the cylinder 321 of the said lower retainer ring 320 is aligned with corresponding holes on other components in said seal membrane assembly 380. Said cylinder 321 and the bore 371 of the upper retainer ring 370 are adopted to interference fit, so that the whole seal membrane assembly 380 is in the compressed state. Said protector assembly 360 is used to protect a center seal body of said seal membrane 330, herein permit the sharp edge of the instrument to pass through without causing perforations or tears.

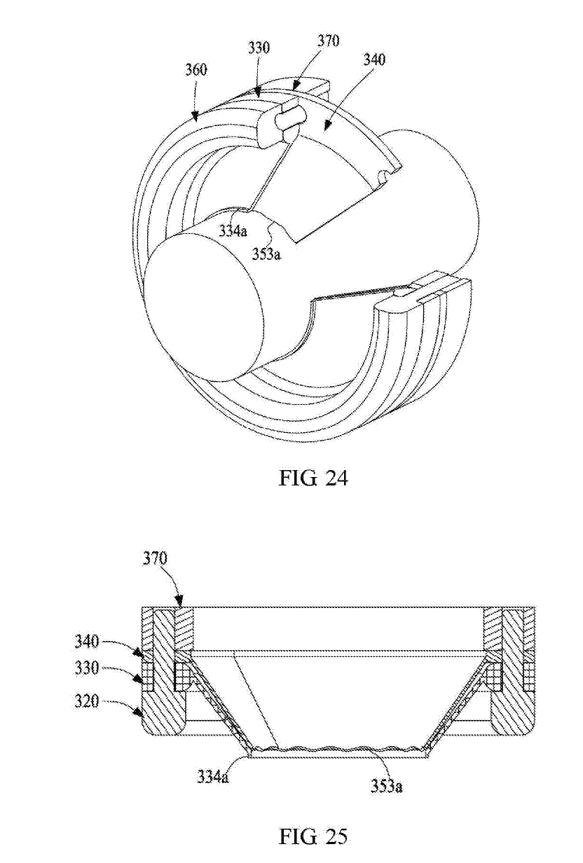

FIG. 24-25 shows a simulated deformation view of said seal membrane assembly 380 which when a large diameter (12.8 mm) instrument is inserted into. Normally, said protector assembly 340 is made a semi-rigid material; or made of rigid material but manifested as semi-rigid due to its thinner thickness; while said seal membrane 330 is made of super elastic material such as silicone, natural rubber or polyisoprene. When a large diameter instrument is inserted, the sealing lip 334 is expanded to be an appropriate sealing lip 334a to accommodate the inserted instrument, and all of the pleats of the protector assembly 340 are simultaneously relaxed to an appropriate size to accommodate the inserted instrument. It will be understood by those skilled in the art that since the protector assembly 340 is semi-rigid, which pleats are generally not entirely relaxed; that is, the relaxed protector assembly 340 still has a smaller annular-wave 353a. If the protector assembly 340 is relaxed and then its annular-wave 353a is close to the sealing lip 334a, it will be easy to cause air leakage or sealing unreliability; and if the annular-wave 353a is far enough away from the sealing lip 334a to ensure its sealing reliability, it will inevitably lead to more sealing walls of the seal membrane 330 exposed, thereby increasing the risk of being pierced or torn, and enlarging the true contact area of the instrument and the seal membrane to certain degree to increase the frictional resistance.

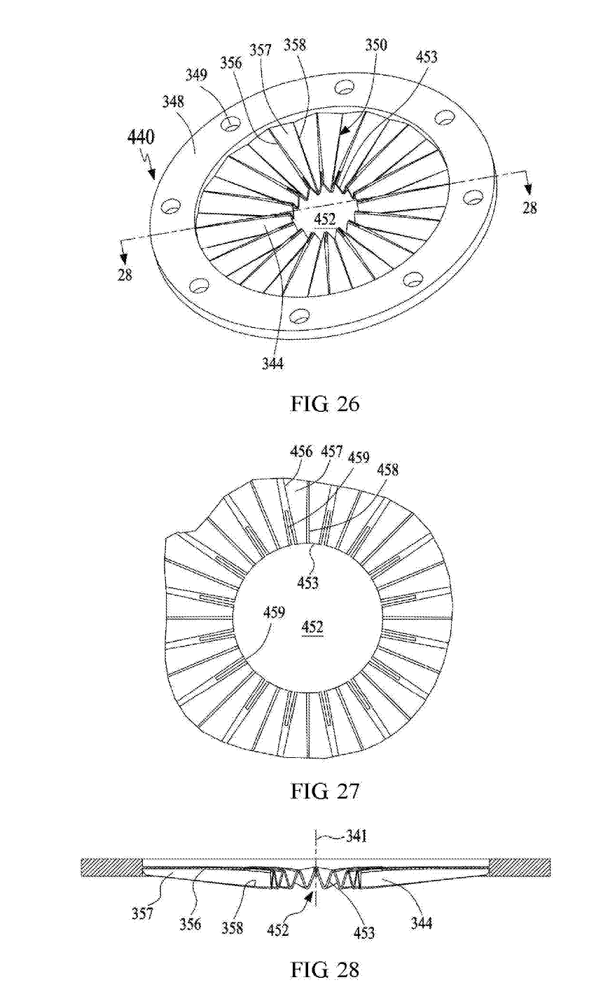

FIG. 26-28 illustrate the structure and composition of the protector assembly 440 in the, second embodiment of the invention. The numerical designations of the geometrical structure in FIG. 26-28 are the same as which in FIG. 18-21 it indicates that the structure of the same designations is basically equivalent. Said protector assembly 440 has a central axis 341, which assembly 440 comprises a proximal end 342 and a distal end 344. The proximal end 342 includes a flange 348 and an aperture 349 generally uniformed over the flange 348. The distal end 344 includes a plurality of pleats 350 that are generally arranged in dish shape around the axis 341 and define a distal aperture 452. More specifically, the distal aperture 452 is defined by a complete annular-wavy line 453, and the annular-wavy line 453 is formed in such a manner that its annular-wave is substantially on a cylindrical surface. Each of the pleats 350 includes a pleated wall 357 extending between the pleat-ridge 356 and the pleat-valley 358. The pleats 350 extend laterally outward from the annular-wavy line 353 and intersect the flange 348.

The protector assembly 440 is substantially similar to shape and structure to the protective device 340, except that the distal aperture 452 of the protector assembly 440 is not defined by the complete annular wavy circuit 453. The protector assembly 440 is not completely seamless in the adjacent area of the distal aperture 452. For the convenience of quantification, it is defined when Di is designed as the maximum diameter of the surgical instrument passing through the seal membrane, taking the axis 341 as the center, draw a cylinder of diameter Di to intersect the distal end 344, the area from its intersection line to the distal 452 is referred to as the aperture-adjacent area. In the present embodiment, the pleat-ridge 356 is cut off by the slot 459 in the adjacent area of the distal aperture 452. The slot 459 can be directly injection molded together with the protector assembly 440, and the width of the slot is as small as possible; the slot 459 can also be processed by secondary processing, for example, if the protector assembly 340 is directly cut, the width of the slot 459 will be close to zero. Although in the present embodiment, in the adjacent area of the distal aperture 452, all of the pleat-ridges contain broken slots; however, the pleat-ridges and the pleat-valleys may be simultaneously slotted, or a portion of the pleat-valleys may be slotted; or a portion of the pleat-ridges may be slotted.

Taking the protector assembly 440 for example, when inserting an instrument(for example, when inserting a titanium applier), since the width of the slot 459 is much smaller than the width of the operating edge of the inserted titanium applier, and since the protector assembly comprises a plurality of pleats, the operating edge of the titanium applier firstly contacts the pleats of the protector assembly and presses to force said partial relaxation of pleats; at this time, the slot 459 generally do not increase, but instead material overlap occurs, and the operating edge of the titanium applier can still be prevented from contacting the sealing wall covered by the protector assembly 440. When the titanium applier is fully inserted into the sealing membrane assembly, the slot 459, in turn, acts to reduce the annular-wave of the adjacent area of the aperture of the protector assembly as described above, and designed as less exposed sealing wall area of the protector assembly, thereby reducing the probability of damage to the seal membrane, the overall relaxation force to a certain extent and frictional resistance generated by the movement of the instrument in the seal membrane. In addition, since the protector assembly 440 is semi-rigid, if the adjacent area of the distal aperture 452 is completely seamless, the circumference of the annular-wavy line 453 must be larger than the outer circumference of the largest diameter instrument designed to be inserted. Whereas when the adjacent area of the distal aperture 452 comprises a plurality of slots 459, the circumference of the annular-wave is not necessarily larger than the outer circumference of the largest diameter instrument designed to be inserted. Therefore, it is possible to reduce the size of the pleats or reduce the number of pleats, thereby simplifying the mold and providing processing efficiency.

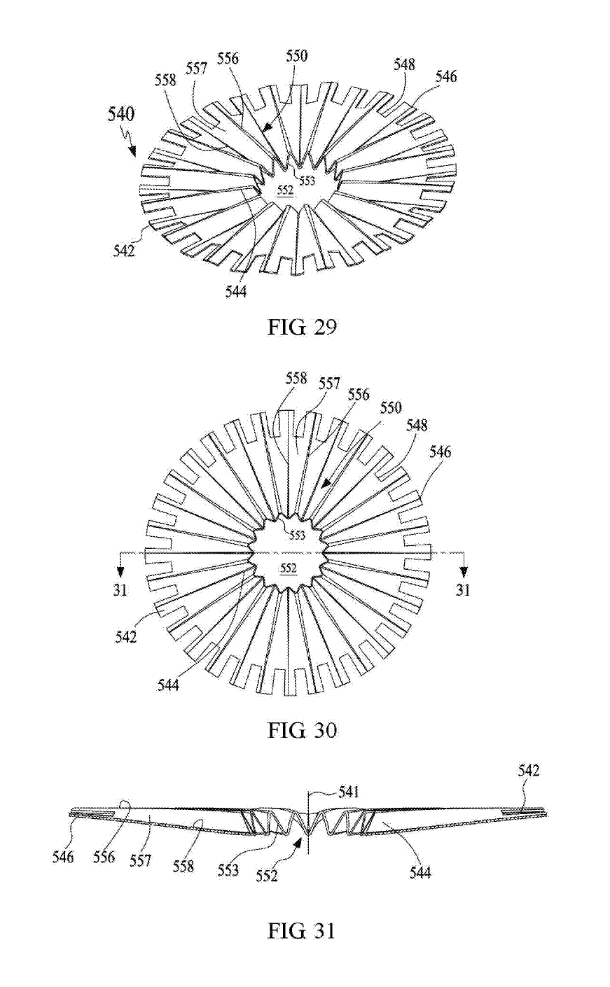

FIG. 29-31 illustrate the structure and composition of the protector assembly 540 in the fifth embodiment of the invention. Said protector assembly 540 has a central axis 541, which assembly 540 comprises a proximal end 542 and a distal end 544. The proximal end 542 includes a plurality of cantilevers 546 and a plurality of indentations 548. The distal end 544 includes a plurality of pleats 550 that are generally arranged in dish shape around the axis 541 and define a distal aperture 552. In the embodiment 16 pleats 550 are included. The distal aperture 552 is defined by a complete annular wavy circuit 553. The distance of the pleat-ridge of the annular wavy circuit 553 relative to the axis 541 is greater than which of its pleat-valleys relative to the axis 541, and the annular wavy circuit 553 is formed in such a manner that its annular-wave is substantially on a conical surface. An arbitrary cylindrical surface is intersected with the pleats with the axis 541 as the center, the intersection line of which is a complete annular wavy circuit; or at least an arbitrary cylindrical surface is intersected with the pleats in the adjacent area of the distal aperture 552 with the axis 541 as the center, the intersection line of which is a complete annular wavy circuit. It is referred to herein as a cylindrical-wavy circuit to distinguish it from the planar-wavy circuit described in the background art. The distal end 511 can be regarded as stacked by numerous complete cylindrical wavy rings with increasing in diameter.

Each of the pleats 550 includes a pleated wall 557 extending from the pleat-ridge 556 to the pleat-valley 158. Said pleats 550 extend laterally outward from annular-wavy line 553, and said pleats 550 extend laterally outward, and the depth of said pleats gradually decrease; the measurement method of said pleats depth is: the distance from the pleat-ridge to the pleat-valley along longitudinal axis 541. One ordinary skilled, in the art will appreciate that the protector assembly 540 can be secured to the seal membrane by gluing, mechanical fastening or welding or otherwise.

Many different embodiments and examples of the invention have been shown and described. One ordinary skilled in the art will be able to make adaptations to the methods and apparatus by appropriate modifications without departing from the scope of the invention. For example, changing the chamfer at the pleat-ridge or the pleat-valley can change the wall thickness at the pleat-ridge or the pleat-valley. As embodiments shown in the invention, the cross-section of the pleats is approximately triangular, but may also be approximately rectangular or trapezoidal. Several modifications have been mentioned, to those skilled in the art, other modifications are also conceivable. Therefore, the scope of the invention should follow the additional claims, and at the same time, it should not be understood that it is limited by the specification of the structure, material or behavior illustrated and documented in the description and drawings.

I claim:

1. An improved trocar seal protector assembly for minimally invasive surgery comprises a proximal opening, a distal end, and a central axis, and the distal end comprises a plurality of pleats, each pleat including a pleat-ridge, a pleat-valley and a pleat-wall extending from the pleat-ridge to the pleat-valley; and the pleats are arranged in a dish-shape around the central axis and define a distal aperture.

2. The seal protector assembly according to claim 1, the distal aperture limits by a cylindrical-wavy circuit.

3. The seal protector assembly according to claim 1, the distal aperture and its adjacent area are formed by stacking of a series of complete cylindrical-wavy circuits.

4. The seal protector assembly according to claim 3, the distal aperture comprising a central axis and a transverse plane substantially perpendicular to the central axis: the angle between the pleat-ridge and the transverse plane surface as α, the angle between the pleat-valley and the transverse plane surface as β, 0°≤α≤15°, 0°≤β≤15°.

5. The seal protector assembly according to claim 3, the protector assembly do not interfere with each other after the inversion, thereby preventing seal membrane and the seal protector assembly from jamming.

6. The seal protector assembly according to claim 3, wherein all or the portion of the pleat-ridge or the pleat-valley includes cuts or slots, in the adjacent area of the distal aperture.

7. The seal protector assembly according to claim 3, wherein the proximal opening of the protector assembly further includes a boss, a cylindrical wall extending from the proximal end to the distal end, and a rib extending from the cylindrical wall and intersecting the pleat-valley; while the pleat-ridge and its adjacent pleat-wall are cantilevered-shape.

8. The seal protector assembly according to claim 7, wherein the proximal, end of the seal protector assembly further includes a mounted flange.

9. A seal membrane assembly, wherein it comprise the protector assembly of claim 8 and a seal membrane; the seal membrane comprise a proximal opening, a flange and a distal aperture, further comprising an outer floating portion extending from the distal aperture to the inner seal body of the flange and extending from the distal aperture to the proximal end; and the flange further including annular inner groove, the boss of the protector assembly is embedded in the annular inner groove.

10. A seal membrane assembly, wherein it comprises said protector assembly of claim 8, a seal membrane, a lower retainer ring and an upper retainer ring; the seal membrane comprises a proximal opening, a flange and a distal aperture, further comprising an outer floating portion extending from the distal aperture to the inner seal body of the flange and extending from the distal aperture to the proximal end; the seal membrane and the protector assembly are sandwiched between the upper retainer ring and the lower retainer ring.
